(19) United States
(12) Patent Application Publication
(10) Pub. No.: US 2014/0107623 A1
(43) Pub. Date: Apr. 17, 2014

(54) Steerable Delivery Sheaths

(71) Applicants: Amr Salahieh, Saratoga, CA (US); Jonah Lepak, Santa Cruz, CA (US); Emma Leung, Santa Cruz, CA (US); Tom Saul, El Granada, CA (US); Jean-Pierre Dueri, Los Gatos, CA (US); Brice Arnault De La Menardiere, Santa Cruz, CA (US); Clayton Baldwin, Santa Cruz, CA (US); Joseph Creagan Trautman, Sunnyvale, CA (US)

(72) Inventors: Amr Salahieh, Saratoga, CA (US); Jonah Lepak, Santa Cruz, CA (US); Emma Leung, Santa Cruz, CA (US); Tom Saul, El Granada, CA (US); Jean-Pierre Dueri, Los Gatos, CA (US); Brice Arnault De La Menardiere, Santa Cruz, CA (US); Clayton Baldwin, Santa Cruz, CA (US); Joseph Creagan Trautman, Sunnyvale, CA (US)

(21) Appl. No.: 14/023,343

(22) Filed: Sep. 10, 2013

Related U.S. Application Data
(60) Provisional application No. 61/699,783, filed on Sep. 11, 2012.

(51) Int. Cl. A61M 25/01

(52) U.S. Cl. CPC A61M 25/0105
USPC 604/528

(57) ABSTRACT

A steerable delivery device comprising an outer sheath and an inner sheath disposed within the outer sheath, wherein the outer sheath includes a first tubular element, a steerable portion adapted to be steered, and wherein in a cross section along the steerable portion the first tubular element includes a first section of a first material with a first durometer and a second section of a second material with a second durometer different than the first durometer.

### CROSS REFERENCE TO RELATED APPLICATIONS

This application claims the benefit of U.S. Provisional App. No. 61/699,783, filed Sep. 11, 2012; and is related to and incorporates by reference herein the following applications: U.S. application Ser. No. 12/823,049, filed Jun. 24, 2010, U.S. application Ser. No. 13/463,537, filed May 3, 2012, and U.S. application Ser. No. 13/463,498, filed May 3, 2012, U.S. Prov. App. No. 61/220,160, filed Jun. 24, 2009, U.S. Prov. App. No. 61/220,163, filed Jun. 24, 2009, U.S. Prov. App. No. 61/232,362, filed Aug. 7, 2009, U.S. Prov. App. No. 61/482,018, filed May 3, 2011, U.S. Prov. App. No. 61/555,687 filed Nov. 4, 2011, U.S. Prov. App. No. 61/555,706, filed Nov. 4, 2011.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIGS. 1A-1C illustrate exemplary inner tubular member 100. FIG. 1A is a top view. FIG. 1B is a view rotated 90 degrees relative to the FIG. 1A view, and FIG. 1C is a view rotated 180 degrees relative to the view in FIG. 1A (and 90 degrees relative to the view in FIG. 1B).

FIGS. 2A-2C illustrate exemplary outer tubular 200 that is part of the delivery device and is disposed outside of and around inner tubular member 100. FIG. 2A is a top view. FIG. 2B is a view rotated 90 degrees from the view in FIG. 2A, and FIG. 2C is a view rotated 180 degrees from the view in FIG. 2A (and 90 degrees from the view in FIG. 2B).

FIGS. 3A-3E illustrate views of assembly 300 including the inner and outer tubular members 100 and 200, respectively, from FIGS. 1 and 2.

### DETAILED DESCRIPTION

The disclosure herein relates generally to steerable delivery devices, and is related to the disclosure of U.S. application Ser. No. 13/463,498, filed May 3, 2012. The exemplary delivery devices described herein are similar to those shown in FIGS. 53A-G in U.S. application Ser. No. 13/463,498. In particular, the inner tubular member of the delivery devices herein is similar to inner tubular member 1652 described in reference to FIGS. 35A-G in U.S. application Ser. No. 13/463,498. The exemplary embodiment in FIGS. 35A-G is described generally in paragraph [00178] in U.S. application Ser. No. 13/463,498.

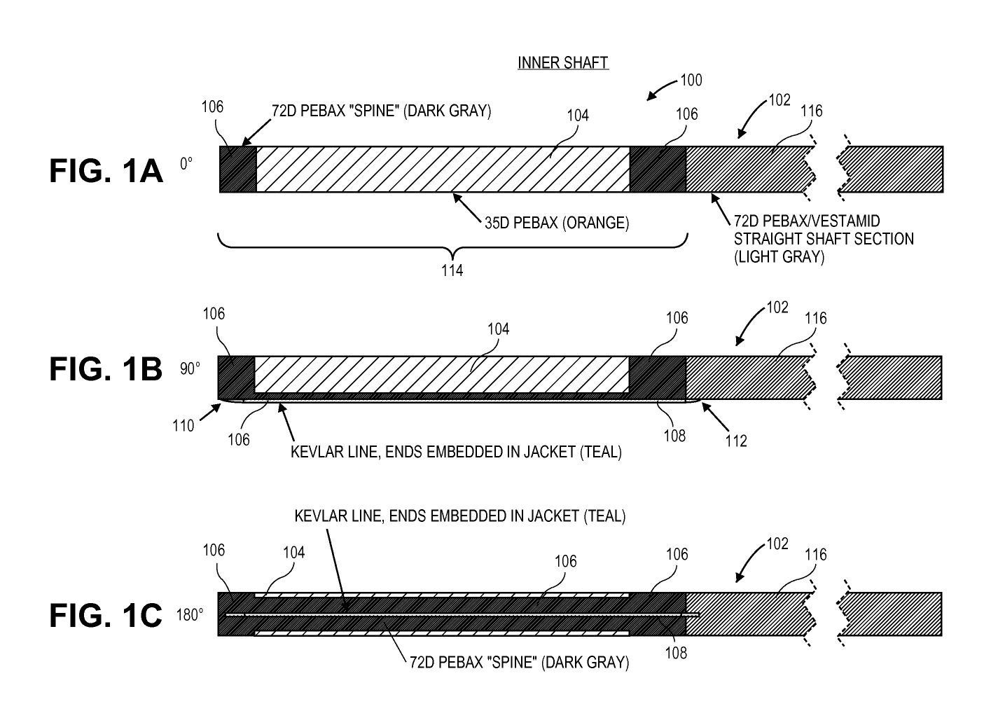

FIGS. 1A-1C illustrate exemplary inner tubular member 100. FIG. 1A is a top view. FIG. 1B is a view rotated 90 degrees relative to the FIG. 1A view, and FIG. 1C is a view rotated 180 degrees relative to the view in FIG. 1A (and 90 degrees relative to the view in FIG. 1B).

Inner tubular member 100 includes steerable distal section 114 and a proximal section 102. Proximal section 102 includes a proximal tubular element 116 with a first durometer. In the embodiment shown proximal tubular element 116 has a durometer of 72D and is a Pebax/vestamid material. Steerable distal section 114 includes tubular element 104 and spine 106. Spine 106 is similar to first portion 1658 from FIGS. 35A-G in U.S. application Ser. No. 13/463,498. Tubular element 104 has a lower durometer than proximal tubular element 116. In this embodiment tubular element 104 has a durometer of 35D, and is Pebax. Spine 106 has optional proximal and distal cuff portions that extend all the way around the device, and a spine section that extends between the two cuff portions that does not extend all the way around the device. In the spine section spine 106 makes up about ¼ of inner tubular member 100, and tubular element 104 makes up about ¾ of the inner tubular member 100. Inner tubular member 100 also includes tensioning member 108 that is secured to the distal end 110 of cuff portion and to the distal end 112 of proximal section 102. Tensioning member 108 is free floating in between the two points at which it is secured. Tensioning member 108 is directly adjacent to, and in alignment with, the spine section of spine 106 (as can be seen in FIG. 1C). In this embodiment tensioning member 108 is a Kevlar line. Spine 106 has a greater durometer than tubular element 104, and in this embodiment is 72D Pebax.

As is described in more detail in U.S. application Ser. No. 13/463,498, the lower durometer of tubular element 104 relative to proximal tubular element 116 allows the steerable distal section to bend. Spine 106, however, due to its higher durometer, reduces shortening in compression and stretching in tension, as can occur in the distal section when it is actuated. For example, the distal section of the inner tubular member may sometimes compress, or shorten, when it is pushed in relative to the outer tubular member to straighten the steerable portion from a bent configuration towards a straighter configuration. The durometers provided are not intended to be limiting but merely illustrative.

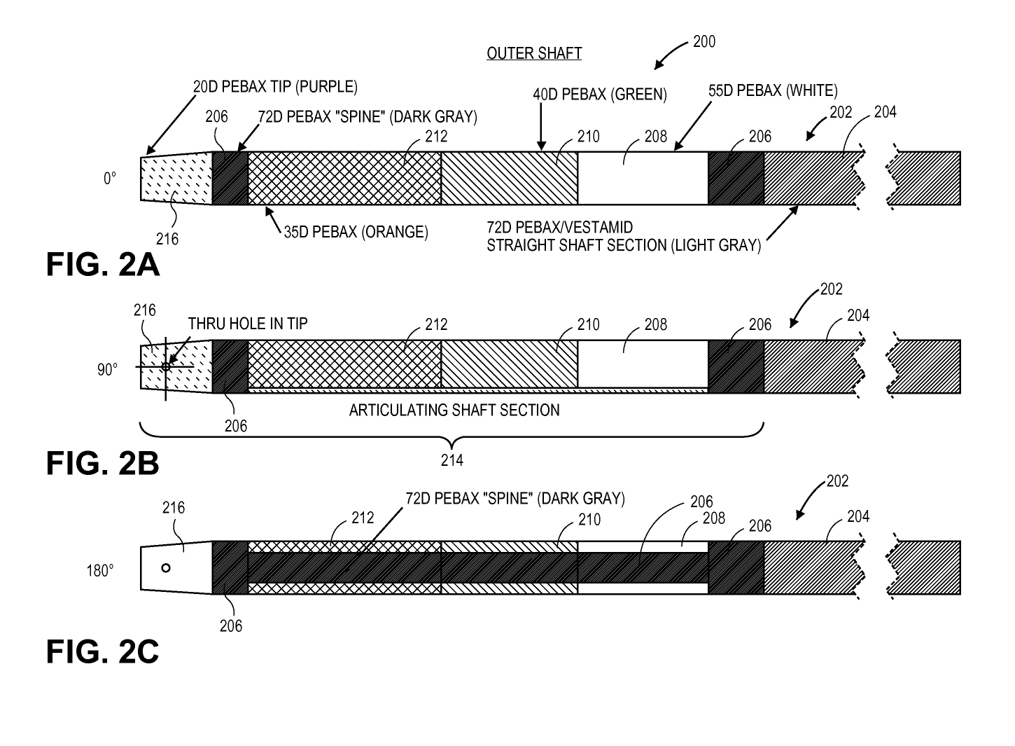

FIGS. 2A-2C illustrate exemplary outer tubular 200 that is part of the delivery device and is disposed outside of and around inner tubular member 100. FIG. 2A is a top view. FIG. 2B is a view rotated 90 degrees from the view in FIG. 2A, and FIG. 2C is a view rotated 180 degrees from the view in FIG. 2A (and 90 degrees from the view in FIG. 2B).

Outer tubular member 200 includes a proximal section 202 and steerable, or articulating, distal section 214. Proximal section 202 has a proximal tubular element 204 with a first durometer. In this embodiment proximal tubular element 204 is a 72D Pebax/Vestamid material. Distal articulating section 214 includes spine 206, which is structurally the same as the spine in FIGS. 1A-1C. Spine 206 includes distal and proximal cuffs and a spine section extending between the two optional cuff portions. In this embodiment spine 206 is 72D Pebax. Articulating section 214 also includes first section 208, second section 210, and third section 212, all of which have different durometers. In this embodiment the durometers decrease towards the distal end of the device. In this embodiment first section 208 is 55D Pebax, second section 210 is 40D Pebax, and third section 212 is 35D Pebax. The multiple bands of different durometer materials (three in this embodiment) in the outer tubular member provide for a more uniform radius of curvature when the steerable section is bent. To the contrary, in embodiment in which the tubular element is all one durometer (excluding the spine), the radius of curvature of the steerable section is generally smallest at the most distal location and increases towards the proximal end. This radius of curvature variation essential forms a spiral in the steerable section. Proximal tubular element 204 has a greater durometer than all three sections 208, 210, and 212. The distal articulating section 214 also includes distal tip 216. In this embodiment distal tip 216 is the lowest durometer material, and in this embodiment is 20D Pebax.

The embodiments herein with the outer spine and the multiple durometer steerable sections provides for advantages in bidirectional use. For example, less force is required to bend the multiple durometer arrangement, hence there is less foreshortening or conversely less stretching when the element is used in tension. This advantage would also hold true for unidirectional steering.

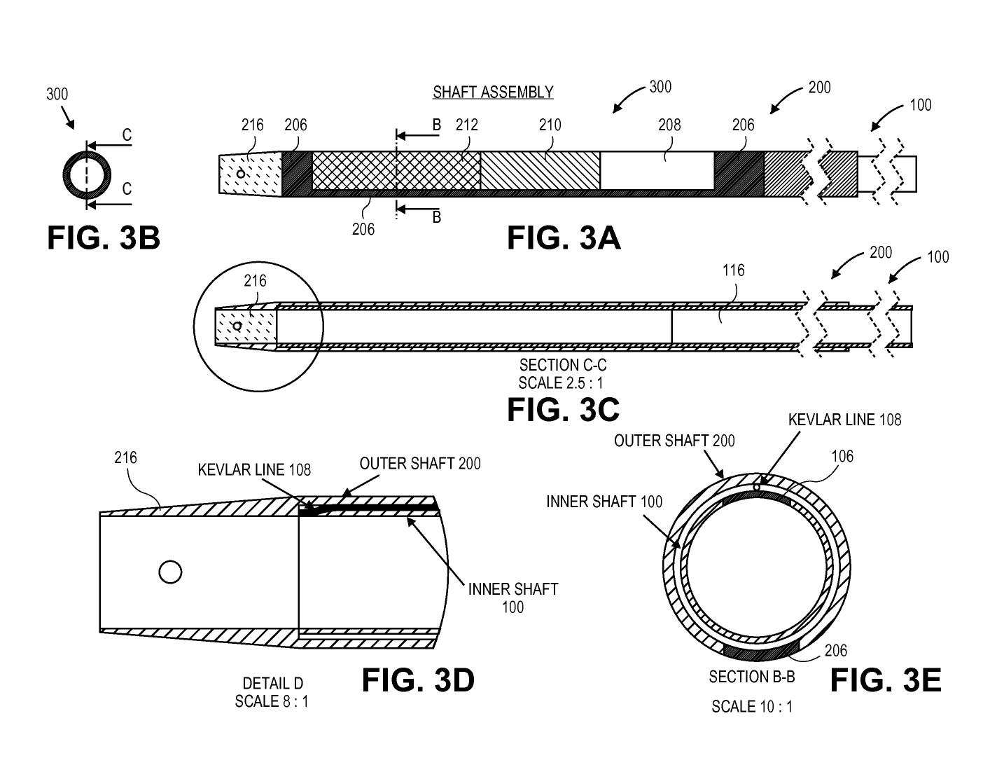

As is described in more detail in the assembly shown in FIGS. 3A-3C, the spines in the inner and outer tubular members are offset 180 degrees from one another. Tensioning member 108 is therefore also offset 180 degrees from the outer spine.

FIGS. 3A-3E illustrate views of assembly 300 including the inner and outer tubular members 100 and 200, respectively, from FIGS. 1 and 2. As can be seen in FIGS. 3A and 3E, tensioning member 108 is offset 180 degrees from outer spine 206. The inner and outer spines are also offset by 180 degrees.

The assembly 300 can be used as is described in the applications incorporated by reference herein. For example, the inner and outer tubular members can be axially moved relative to one another to steer the distal steerable section. When a spine from one tubular member is put in tension, the other spine is put in compression. The dual spine embodiment reduces shortening in one tubular member in compression and stretching in the other tubular member in tension.

In some embodiments the inner or outer tubular members are formed by positioning the different materials on a mandrel, placing shrink wrap over the different materials, and increasing the temperature, which causes the material to melt together, forming the inner or outer tubular members. The optional cuffs described above can be helpful in securing one or more components together during the manufacturing process.

Any of the inner and outer tubular members described in U.S. application Ser. No. 13/463,498, filed May 3, 2012 that comprise one or more slots or spines can be made of an elastomeric or polymeric material. For example, in U.S. application Ser. No. 13/463,498, the tubular members shown in FIG. 2, 3, or 4 with slots and spines therein can be made from Pebax.

What is claimed is:

1. A steerable delivery device comprising:
an outer sheath and an inner sheath disposed within the outer sheath, wherein the outer sheath includes a first tubular element;
a steerable portion adapted to be steered;
wherein in a cross section along the steerable portion the first tubular element includes a first section of a first material with a first durometer and a second section of a second material with a second durometer different than the first durometer.

2. A steerable delivery device comprising:
an outer sheath and an inner sheath disposed within the outer sheath, wherein the inner sheath includes a first tubular element and the outer sheath includes a second tubular element;
a steerable portion adapted to be steered;
wherein in a cross section along the steerable portion the first tubular element includes a first section of a first material with a first durometer and a second section of a second material with a second durometer different than the first durometer and the second tubular element includes a first section of a first material with a first durometer and a second section of a second material with a second durometer different than the first durometer.
